(19) United States
(12) Patent Application Publication
(10) Pub. No.: US 2025/0268888 A1
(43) Pub. Date: Aug. 28, 2025

(54) BRONCHODILATORS FOR TREATING OBSTRUCTIVE LUNG DISEASE

(71) Applicants: University of South Florida, Tampa, FL (US); Rutgers, The State University of New Jersey, New Brunswick, NJ (US)

(72) Inventors: Stephen Liggett, Tampa, FL (US); James Leahy, Tampa, FL (US); Donghwa Kim, Tampa, FL (US); Steven An, Highland Park, NJ (US)

(21) Appl. No.: 19/069,790

(22) Filed: Mar. 4, 2025

Related U.S. Application Data
(63) Continuation of application No. 17/464,233, filed on Sep. 1, 2021, now Pat. No. 12,257,248.
(60) Provisional application No. 62/706,654, filed on Sep. 1, 2020.

(51) Int. Cl. A61K 31/4745; A61K 31/122; A61K 31/444

(52) U.S. Cl. CPC A61K 31/4745; A61K 31/122; A61K 31/444

(57) ABSTRACT

Disclosed herein are compounds useful for treating obstructive lung diseases. The compounds are TAS2Rs agonists and may further be used to treat disorders and conditions implicated by TAS2R. In some instances the disclosed compounds can be used to treat asthma.

### CROSS-REFERENCE TO RELATED APPLICATION

This application is a continuation of U.S. application Ser. No. 17/464,233, filed Sep. 1, 2021, which claims the benefit of U.S. Provisional Application 62/706,654, filed Sep. 1, 2020, the contents of which are hereby incorporated in their entirety.

### ACKNOWLEDGEMENT OF GOVERNMENT SUPPORT

This invention was made with government support under grant/contract HL114471 awarded by the National Institutes of Health. The government has certain rights in the invention.

### FIELD OF THE INVENTION

The invention is directed to compounds, compositions, and methods for treating obstructive lung diseases, including asthma.

### BACKGROUND

Bitter taste receptors (TAS2Rs) are members of the G protein coupled receptor (GPCR) superfamily of receptors, and traditionally were thought to have expression confined to taste cells where they detect bitter substances. The human genome encodes 25 TAS2R subtypes, with varying degrees of specificities to bitter tastants. Some subtypes are described as being “broadly tuned” while others appear to have highly restrictive requirements for agonist binding and activation. Recent studies have shown that TAS2Rs are expressed on multiple cell types and tissues throughout the body, representing a previously unrecognized chemosensory system responding to endogenous and exogenous substances, including potential novel therapeutic targets.

Of particular interest has been the delineation of six TAS2R subtypes (TAS2Rs 4, 5, 10, 14, 19, 31) that are ex-pressed on human airway smooth muscle (HASM) cells. TAS2R activation in these cells results in coupling of the receptor to the Gαi family of G proteins with a subsequent increase in intracellular Ca²⁺ ([Ca^{2+]}ᵢ) in microdomains that leads to marked HASM relaxation. This has prompted investigations into the use of TAS2R agonists as a potential treatment for obstructive lung diseases such as asthma, where HASM contraction is a main mechanism of airflow restriction. Five of the aforementioned six TAS2R subtypes appear to be broadly tuned, being activated by compounds with a wide-ranging structural properties, albeit with typically low apparent affinities. These properties may be due to the evolution of TAS2Rs on taste cells in order to detect the large number bitter tasting toxic substances found in plants. This diversity is illustrated by studies with TAS2R14, which show that it is activated by quinine, aristolochic acid, chlorhexidine, and flufenamic acid, as well as >29 other known compounds. Furthermore, many of these agonists also activate other TAS2R sub-types not expressed on HASM, but found in other organs such as heart, thyroid, pancreas, and uterus. For drug development, this lack of more specific high-affinity binding requirements of TAS2R binding pockets for activation represents a challenge for identifying receptor ligands for therapeutic purposes.

Currently, the only available direct bronchodilators are β₂-adrenergic receptor (β₂AR) agonists, also called “β-agonists”. These agents activate the cell surface β₂AR on HASM and activate Gαs, stimulating adenylyl cyclase and increasing cellular cAMP and activation of protein kinase A, which leads to relaxation. β-agonists are associated with a number of adverse effects, and about one-half of asthmatics fail to reach optimal control. Thus, efforts to find new classes of direct bronchodilators targeting a number of HASM proteins are underway. These include not only TAS2Rs, but Rho kinase inhibitors, prostanoid receptor agonists, peroxisome proliferator-activated receptor y agonists and pepducins that modify GPCR signaling. Our studies of HASM TAS2Rs for potential new bronchodilator targets are based on experiments where we find a high level of efficacy for relaxation of human bronchi, no effect of the asthma cellular phenotype on TAS2R function, additive effects with β-agonists without cross-desensitization, and their distinct mechanism of action.

The remains a need for improved bronchodilating agents. There remains a need for improved TAS2R5 agonists. There remains a need for improved methods of treating obstructive lung disease, including asthma.

### SUMMARY

In accordance with the purposes of the disclosed materials and methods, as embodied and broadly described herein, the disclosed subject matter, in one aspect, relates to compounds, compositions and methods of making and using compounds and compositions. In specific examples, the compounds have the formula:

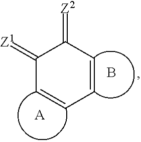

or a pharmaceutically acceptable salt thereof, wherein
ring A represents a carbocyclic or heterocyclic ring system;
ring b represents a carbocyclic or heterocyclic ring system;
Z¹ is O, S, or NR^{a}; wherein R^{a} is H, C₁₋₈alkyl, or C₁₋₈alkoxy; and
Z² is O, S, or NR^{b}; wherein R^{b} is H, C₁₋₈alkyl, or C₁₋₈alkoxy.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts the general signaling paradigm of TAS2Rs in airway smooth muscle cells. Agonist binds to residues in a pocket formed by the hydrophobic transmembrane domains of the receptor, resulting in a conformation that binds and activates the heterotrimeric G protein Gᵢ, ultimately causing an increase in [Ca²⁺]ᵢ.

FIG. 2 depicts the chemical structures of compounds screened for [Ca²⁺]ᵢ stimulation and relaxation in HASM cells.

FIG. 3 depicts Concentration-response curves of compounds screened for stimulation of [Ca²⁺]ᵢ in D9-HASM cells. Results are mean±SE from 4-8 experiments.

FIG. 4 depicts representative [Ca²⁺]i responses in D9-HASM cells over 100 seconds after exposure to the indicated compounds.

FIG. 5 depicts [Ca²⁺]ᵢ responses in primary HASM cells with T5-1 and T5-8. Cultured primary HASM cells at passages 5-7 derived from a non-asthmatic lung were exposed to the indicated concentrations of the agonists. Results are from 4 independent experiments. *, EC50 P<0.01 vs T5-1.

FIG. 6 depicts HASM relaxation responses to non-TAS2R5 agonists measured by MTC. Cells were exposed to the beta-agonists albuterol (partial agonist), formoterol (full agonist), and the direct activator of adenylyl cyclase, forskolin. Results are from 200-300 measurements per condition. *, peak maximal response less than formoterol, P<0.01.

FIG. 7 depicts HASM cell relaxation responses to selected compounds. Compounds were studied at a concentration of 500 μM and were added to the culture media at the 60 second time point (arrow). Results are from measurements of 200-400 cells per compound. *, P<0.01 vs the T5-1 reference response. As can be seen, compound 8 gave the greatest degree of relaxation, followed by 3, then 15, then 18 and 11.

FIG. 8 depicts the same information as FIG. 7, but with additional identifying information for each data stream.

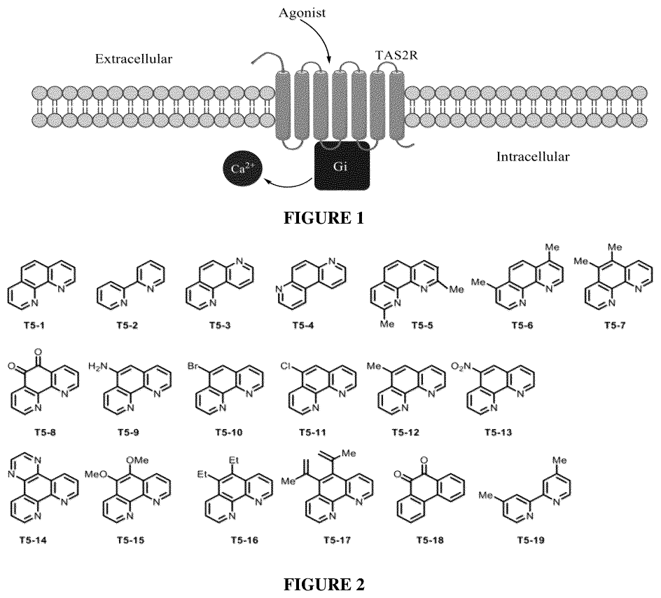

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used herein “aromatic” refers to an unsaturated cyclic molecule having 4n+2π electrons, wherein n is any integer. The term “non-aromatic” refers to any saturated system or unsaturated cyclic molecule which does not fall within the definition of aromatic.

The term “acyl” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkyl” or “alkyl group” refers to a fully saturated, straight or branched hydrocarbon chain radical having from one to forty carbon atoms, and which is attached to the rest of the molecule by a single bond. Alkyls comprising any number of carbon atoms from 1 to 20 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Alkenyl group comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C9 and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” refers to the group —OR, where R is alkyl, alkenyl, alkynyl, cycloalkyl, or heterocyclyl as defined herein. Unless stated otherwise specifically in the specification, alkoxy can be optionally substituted.

“Alkylcarbamoyl” refers to the group −O−C(O)—NRₐR_{b}, where Rₐ and R_{b} are the same or different and independently an alkyl, alkenyl, alkynyl, aryl, heteroaryl, cycloalkyl, or heterocyclyl group, as defined herein, or RₐR_{b} can be taken together to form a heterocyclyl group, as defined herein. Unless stated otherwise specifically in the specification, alkylcarbamoyl can be optionally substituted.

“Alkylcarboxamidyl” refers to the group —C(O)—NRₐR_{b}, where Rₐ and R_{b} are the same or different and independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, alkylcarboxamidyl can be optionally substituted.

“Alkoxycarbonyl” refers to the group —C(O)OR, where R is alkyl, alkenyl, alkynyl, aryl, heteroaryl, cycloalkyl, cycloalkenyl, cycloalkynyl, or heterocyclyl group, as defined herein. Unless stated otherwise specifically in the specification, alkoxycarbonyl can be optionally substituted.

“Alkylthio” refers to the —SR or —S(O)ₙ₌₁₋₂-—, where R is alkyl, alkenyl, alkynyl, cycloalkyl, cycloalkenyl, cycloalkynyl, or hetereocyclyl, as defined herein. Unless stated otherwise specifically in the specification, alkylthio can be optionally substituted.

“Arylthio” refers to the —SR or —S(O)ₙ₌₁₋₂—R, where R is aryl or hetercoaryl, as defined herein. Unless stated otherwise specifically in the specification, arylthio can be optionally substituted.

“Alkynyl” or “alkynyl group” refers to a straight or branched hydrocarbon chain radical having from two to forty carbon atoms, and having one or more carbon-carbon triple bonds. Each alkynyl group is attached to the rest of the molecule by a single bond. Alkynyl group comprising any number of carbon atoms from 2 to 40 are included. An alkynyl group comprising up to 40 carbon atoms is a C₂-C₄₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain, having from two to forty carbon atoms, and having one or more carbon-carbon triple bonds. Non-limiting examples of C₂-C₄₀ alkynylene include ethynylene, propargylene and the like. Unless stated otherwise specifically in the specification, an alkynylene chain can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Unless stated otherwise specifically in the specification, the carbocyclyl can be a monocyclic, bicyclic, tricyclic or tetracyclic ring system, which can include fused or bridged ring systems Carbocyclic rings include cycloalkyl, cycloalkenyl, and cycloalkynyl as defined herein. In some embodiments, the carbocyclyl is monovalent and is attached to the rest of molecule through a single bond. In some embodiments, the carbocyclyl is divalent and is independently attached to two moieties through single bonds. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo [2.2.1] heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Heterocyclyl,” “heterocyclic ring” or “heterocycle” refers to a stable 3-to 20-membered ring radical, which consists of two to fourteen carbon atoms and from one to eight heteroatoms selected from the group consisting of nitrogen, oxygen and sulfur. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be aromatic, partially saturated, or fully saturated. Examples of heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopipcridinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl is monovalent and is attached to the rest of molecule through a single bond. In some embodiments, the heterocyclyl is divalent and is independently attached to two moieties through single bonds. Unless stated otherwise specifically in the specification, a heterocyclyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from accanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aryloxy” refers to groups -OAr, where Ar is an aryl or heteroaryl group as defined herein. Unless otherwise stated specifically in the specification, the aryloxy group can be optionally substituted.

“Heteroaryl” refers to a 5-to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Aralkyl” refers to a radical of the formula —R_{b}—R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double-or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced

with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(50 O)OR_{g}, —C(50 O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Unless stated to the contrary, a substituent drawn without explicitly specifying the point of attachment indicates that the substituent may be attached at any possible atom. For example, in a benzofuran depicted as:

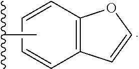

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

As used herein, the term “about” or “approximately” refers to a quantity, level, value, number, frequency, percentage, dimension, size, amount, weight or length that varies by acceptable levels in the art. In some embodiments, the amount of variation may be as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

A numerical range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Disclosed herein are bronchodilating compounds and methods of using the disclosed bronchodilating compounds in the treatment of obstructive lung diseases. In some embodiments, the compounds have the formula:

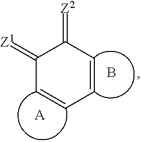

or a pharmaceutically acceptable salt thereof, wherein
ring A represents a carbocyclic or heterocyclic ring system;
ring b represents a carbocyclic or heterocyclic ring system;
Z¹ is O, S, or NR^{a}; wherein R^{a} is H, OH, C₁₋₈alkyl, or C₁₋₈alkoxy; and
Z² is O, S, or NR^{b}; wherein R^{b} is H, OH, C₁₋₈alkyl, or C₁₋₈alkoxy.

In some embodiments, Z¹ and Z² are each O. In other embodiments, Z¹ is NR^{a} and Z² is O. In other embodiments, Z² is NR^{a} and Z¹ is O. In further embodiments Z¹ and Z² are each NR^{a}. Exemplary R^{a} groups include H, OH, C₁₋₂alkyl, and C₁₋₂alkoxy.

In some embodiments, ring A can include an sp² hybridized nitrogen atom bonded to the central ring para to the Z² bearing carbon atom. Such compounds may be depicted by the formula:

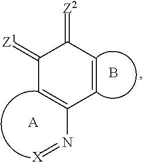

wherein
X is N or CR¹, wherein R¹ is selected from R^{a}, OR^{a}, N(R^{a})₂, F, Cl, Br, I, NO₂, CN, wherein R^{a} is in each case selected from H, C₁₋₈alkyl, C₂₋₈alkenyl; C₂₋₈alkynyl. Suitable ring A systems include:

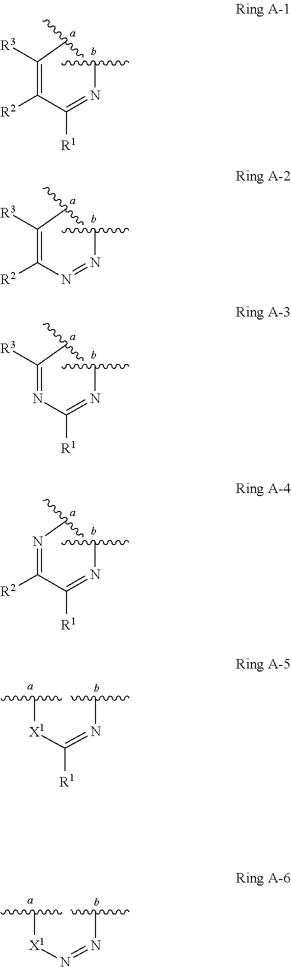

wherein wavy line “a” represent the point of attachment to the carbon ortho to the Z¹ bearing carbon, and wavy line “b” represents the point of attachment to the carbon para to the Z² bearing carbon;
X¹ is O, S, or NR⁴
R² is selected from R^{b}, OR^{b}, N(R^{b})₂, F, Cl, Br, I, NO₂, CN, wherein R^{b} is in each case selected from H, C₁₋₈alkyl, C₂₋₈alkenyl; C₂₋₈alkynyl;
R³ is selected from R^{c}, OR^{c}, N(R^{c})₂, F, Cl, Br, I, NO₂, CN, wherein R^{c} is in each case selected from H, C₁₋₈alkyl, C₂₋₈alkenyl; C₂₋₈alkynyl;
R⁴ is H or C₁₋₈alkyl, C₂₋₈alkenyl; C₂₋₈alkynyl;
wherein any two or more of R¹, R², R³, and R⁴ may together form a ring; and
wherein either R³ or R⁴ may form a ring with R^{a}.

In some embodiments ring B can be an aromatic or heteroaromatic system having the formula:

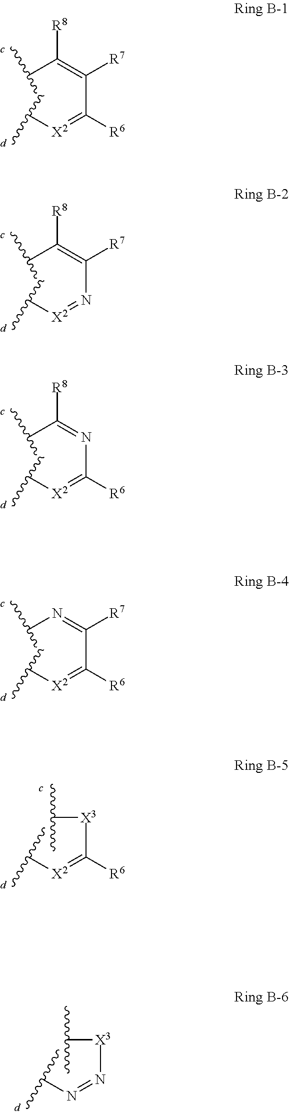

wherein wavy line “c” represent the point of attachment to the carbon ortho to the Z² bearing carbon, and wavy line “d” represents the point of attachment to the carbon para to the Z¹ bearing carbon;
X² is N or CR⁵;
X³ is N or CR⁹;
R⁵ is H or C₁₋₈alkyl, C₂₋₈alkenyl; C₂₋₈alkynyl;
R⁹ is H or C₁₋₈alkyl, C₂₋₈alkenyl; C₂₋₈alkynyl;
R⁶ is selected from R^{d}, OR^{d}, N(R^{d})₂, F, Cl, Br, I, NO₂, CN, wherein R^{d} is in each case selected from H, C₁₋₈alkyl, C₂₋₈alkenyl; C₂₋₈alkynyl;
R⁷ is selected from R^{e}, OR^{e}, N(R^{e})₂, F, Cl, Br, I, NO₂, CN, wherein R^{e} is in each case selected from H, C₁₋₈alkyl, C₂₋₈alkenyl; C₂₋₈alkynyl;
R⁸ is selected from R^{f}, OR^{f}, N(R^{f})₂, F, Cl, Br, I, NO₂, CN, wherein R^{f} is in each case selected from H, C₁₋₈alkyl, C₂₋₈alkenyl; C₂₋₈alkynyl;
wherein any two or more of R⁵, R⁶, R⁷, and R⁸ may together form a ring; and
wherein either R⁸ or R⁹ may form a ring with R^{b}.

Exemplary compounds include those in which Z¹ and Z² are each O, ring A is Ring A-1, and ring B is Ring B-1, wherein X² is N or CH. In other embodiments, ring A is Ring A-1, and ring B is Ring B-2, wherein X² is N or CH. In other embodiments, ring A is Ring A-1, and ring B is Ring B-3, wherein X² is N or CH. In other embodiments, ring A is Ring A-1, and ring B is Ring B-4, wherein X² is N or CH.

In some embodiments, the compound has the formula:

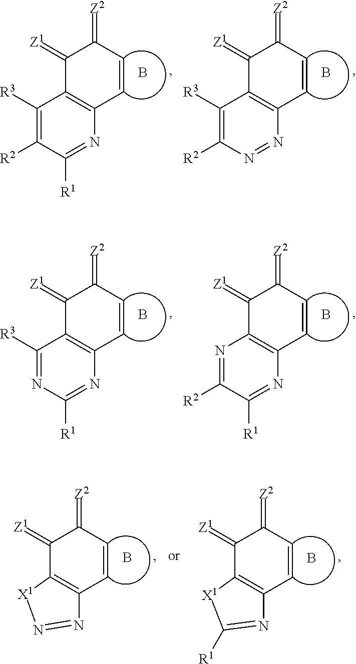

wherein Z¹, Z², ring B, R¹, R², R³, and X¹ are as defined above. Preferably, Z¹ and Z² are each O

In some embodiments, the compound has the formula:

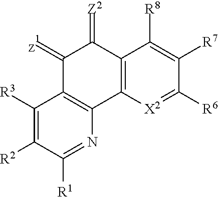

wherein Z¹, Z², R¹, R², R³, R⁶, R⁷, R⁸, and X² are as defined above

In certain embodiments, ring B is Ring B-1, B-2, or B-3, and Z² is NR^{a}. In further embodiments, R^{a} can together form a ring with R⁸. Exemplary systems include:

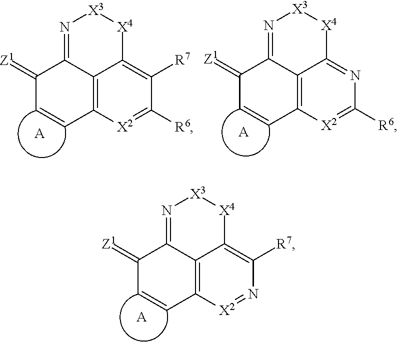

wherein ring A, Z¹, X², R⁶, and R⁷ are as defined above, and X³ is selected from null, O, C₁₋₃alkylene, and X⁴ is selected from null, O, C₁₋₃alkylene, provided that X³ and X⁴ are not both null, and also provided that X³ and X⁴ are not both O.

In certain embodiments, ring A is Ring A-1, A-2, or A-3, and Z¹ is NR^{a}. In further embodiments, R^{a} can together form a ring with R³. Exemplary systems include:

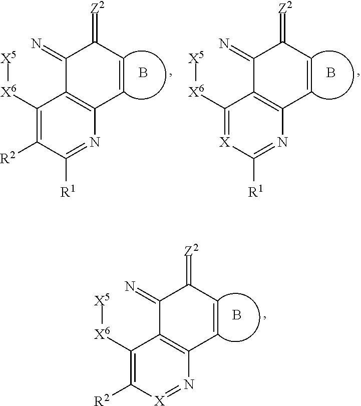

wherein ring B, Z², X, R¹, and R² are as defined above, and X⁵ is selected from null, O, C₁₋₃alkylene, and X⁶ is selected from null, O, C₁₋₃alkylene, provided that X³ and X⁴ are not both null, and that X³ and X⁴ are not both O.

It is further contemplated that in some embodiments, both Z¹ and Z² are NR^{a}, and each R^{a} forms a ring with R³ and R⁸, e.g.,

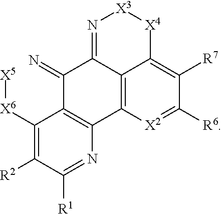

Also provided herein are methods of use of the compounds or compositions described herein. Also provided herein are methods for treating a disease or pathology in a subject in need thereof comprising administering to the subject an effective amount of any of the compounds or compositions described herein.

Disclosed are methods of treating an obstructive lung disease or condition in a subject, comprising administering a therapeutically effective amount of a pharmaceutical composition comprising one or more compounds disclosed herein to a subject in need of treatment. In particular aspects, the obstructive lung disease or condition can be, e.g., asthma, chronic obstructive pulmonary disease (COPD), emphysema or bronchitis.

In a second aspect, provided herein are methods of inducing bronchodilation in a subject, comprising administering a therapeutically effective amount of a pharmaceutical composition comprising one or more compounds disclosed herein to a subject in need of bronchodilation.

In a third aspect, provided herein are methods of relaxing airway smooth muscle (ASM) in a subject, comprising administering a therapeutically effective amount of a pharmaceutical composition comprising one or more compounds disclosed herein to a subject in need of ASM relaxation.

In a fourth aspect, provided herein are methods of treating or preventing bronchoconstriction or bronchospasm in a subject, comprising administering a therapeutically effective amount of a pharmaceutical composition comprising one or more compounds disclosed herein to a subject in need of treatment or prevention of bronchoconstriction or bronchospasm.

The obstructive lung diseases and conditions encompassed by the present invention include any respiratory condition or disease, whether acute or chronic, characterized by impairment of airflow into and/or out of the lungs of a subject. Obstructive lung diseases and conditions include, e.g., asthma, chronic obstructive pulmonary disease (COPD), emphysema and bronchitis, as well as cystic fibrosis, bronchiectasis, bronchiolitis, and allergic bronchopulmonary aspergillosis. Another such obstructive lung disease or condition that can be treated or prevented by administering compounds as described herein includes bronchoconstriction or bronchospasm that can be caused, e.g., by inhalation of a noxious compound such as smoke or a corrosive chemical; by a respiratory infection; or by anaphylaxis such as that caused by sepsis or an allergic reaction to a food (e.g., peanuts), a drug (e.g., penicillin), an insect sting or bite, pollen, mold, dust mites, latex, or other substances; or by other triggers of bronchoconstriction or bronchospasm. For example, the compounds can be administered to prevent (or treat) bronchospasm induced by exercise or air pollution. In another example, the compounds can be administered before or during placement of a breathing tube to prevent (or treat) bronchospasm induced by placement of the tube. The compounds of the invention can be administered to healthy individuals in situations in which it might be desirable to increase bronchodilation to improve oxygen uptake, e.g., in lower oxygen environments (such as several thousand feet above sea level) or to improve athletic performance.

The pharmaceutical compositions of the present invention comprising one or more compounds disclosed herein may also comprise one or more of a carrier, diluent and excipient (e.g., a pharmaceutically acceptable carrier, diluent, or excipient), depending on the identity of the compound. By “pharmaceutically acceptable” is meant a material that is not biologically or otherwise undesirable, i.e., the material may be administered to an individual along with the compound without causing any undesirable biological effects or interacting in a deleterious manner with any of the components of the pharmaceutical composition in which it is contained. Conventional pharmaceutical practice may be employed to provide suitable formulations or compositions to administer such compositions to subjects. The terms specifically exclude cell culture medium. Suitable diluents (for both dry and liquid pharmaceutical formulations) are well known to those skilled in the art and include saline, buffered saline, dextrose (e.g., 5% dextrose in water), water, glycerol, ethanol, propylene glycol, polysorbate 80 (Tween-80™), poly(ethylene) glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin; see, e.g., U.S. patent application publication 20060194717).

Carriers are compounds and substances that improve and/or prolong the delivery of an active ingredient to a subject in the context of a pharmaceutical formulation. Carriers may serve to prolong the in vivo activity of a drug or slow the release of the drug in a subject, using controlled-release technologies. Carriers may also decrease drug metabolism in a subject and/or reduce the toxicity of the drug. Carriers can also be used to target the delivery of the drug to particular cells or tissues in a subject. Common carriers (both hydrophilic and hydrophobic carriers) include fat emulsions, lipids, PEGylated phospholids, liposomes and lipospheres, microspheres (including those made of biodegradable polymers or albumin), polymer matrices, biocompatible polymers, protein-DNA complexes, protein conjugates, erythrocytes, vesicles and particles.

Excipients included in a pharmaceutical composition have different purposes depending, for example on the nature of the drug, and the mode of administration. Examples of generally used excipients include, without limitation: stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, administration aids, and combinations thereof.

The pharmaceutical compositions of the present invention can be formulated for pulmonary administration, whether for nasal or buccal inhalation. The unit dosage of the pharmaceutical composition may be conveniently delivered in the form of an aerosol spray from a pressurized pack or a nebulizer, or via a vaporizer. The pharmaceutical compositions may also be delivered as a formulated powder and the powder composition may be inhaled with the aid of an insufflation powder inhaler device. One example of a delivery system for inhalation is a metered dose inhalation (MDI) aerosol, which may be formulated as a dry suspension or wet solution of a pharmaceutical composition of the invention in a suitable propellant, such as a fluorocarbon solvent or a hydrocarbon solvent. Suitable fluorocarbon solvents include HFA-134a (1,1,1,2-tetrafluorocthanc), HFA-227ea (1,1,1,2,3,3,3-heptafluoropropane), HFA-152a (1,1-difluoroethane) and combinations thereof. For propellant formulations, the propellant may be present in an amount that is at least 5% by weight, at least 10% by weight, at least 25% by weight, at least 50% by weight at least 75% by weight, or at least 90% by weight, relative to the entire formulation. In some embodiments, the propellant is present in an amount from 80-99.9% by weight, or from 90-99.9% by weight, relative to the entire formulation. The propellant formulation may also include one or more stabilizing excipients, such as ethanol and oleic acid. When ethanol is used, it may be present in an amount from 0.5-10% by weight, from 1-5% by weight, or from 5-10% by weight, relative to the entire formulation. The propellant formulations may further include one or more surfactants, for instance in an amount from 0.1-2.5% by weight, or from 0.2-1.5% by weight, relative to the entire formulation.

The compounds may be in particulate form to enhance delivery to the lung. For example, the compounds may be provided with a particle from 0.1-10 μm, from 0.1-5 μm, from 0.5-2.5 μm, from 1-5 μm, from 2.5-5 μm, or from 1-2.5 μm. In the case of dry powder formulations, the disclosed compounds may be provided in an amount from 1-50% by weight, from 1-50% by weight, from 5-50% by weight, from 1-25% by weight, from 10-25% by weight, from 15-50% by weight, or from 25-50% by weight relative to the total weight of the formulation. The dry powder formulations may further include a powdered matrix material, for example a polyol or carbohydrate, e.g., sorbitol, mannitol, xylitol, glucose, arabinose, lactose, maltose, saccharose, dextrose and mixtures thereof. The compositions may further include a surfactant, for example in an amount from 0.1-10% by weight, from 0.1-5% by weight, 1-10% by weight, from 1-5% by weight, 2.5-10% by weight, or from 2.5-5% by weight. Suitable surfactants include lecithin, phospholipids derivatives such as phosphatic acids, phosphatidyl choline (saturated and unsaturated), phosphatidyl ethanol amine, phosphatidyl glycerol, phosphatidyl serine, phosphatidyl inositol, diolcoylphosphatidylcholine, dimyristoyl phosphatidylcholine, dipalmitoylphosphatidylcholine, distearoyl phosphatidylcholine, diarachidoyl phosphatidylcholine, dibenoyl phosphatidylcholine, ditricosanoyl phosphatidylcholine, dilignoceroylphatidylcholine, dimiristoylphosphatidylethanol-amin, dipalmitoyl-phosphatidylethanoalamine, pipalmitoleoylphasphatidylethanol-amine, distearoyl-phosphatidylethanolamine, dimyristoylphosphatidylglycerol, dipalmitoylphosphatidyl glycerol, dipalmitolcoylphosphatidylglycerol, and mixtures thereof.

In addition, any other appropriate route for administration can be employed, for example, but not limited to, intravenous, parenteral, transbuccal, transdermal, transcutaneous, subcutaneous, intranasal, aerosol, or oral administration. Therapeutic formulations may be in the form of liquid solutions or suspensions; for example, for oral administration, formulations may be in the form of tablets or capsules; for intranasal formulations, in the form of powders, nasal drops, or aerosols; for transdermal formulations, in the form of creams or distributed onto patches to be applied to the skin.

Effective amounts of the one compounds disclosed herein in a pharmaceutical formulation will vary depending on the compound being used and the condition or disease being treated, as well as factors such as age of the subject and other medications being taken. Effective dosages will typically be set by an attending physician as is well known in the art. However, the concentration of the compounds delivered to a subject in a unit dose will generally range from about 0.05 mg to about 100 mg, or a value within this range. The combinations can be administered in combinations and/or in combination with one or more other agent (e.g. but not limited to a beta-agonist such as albuterol). In one non-limiting example provided herein, the compounds can be administered with either isoproterenol or chloroquine is additive; thus, under some circumstances, it can be appropriate to administer either (or both) of these agents combination with the compounds disclosed herein to a subject in need thereof.

As used herein, the terms “dose”, “dosage”, “unit dose”, “unit dosage”, “effective dose” and related terms refer to physically discrete units that contain a predetermined quantity of active ingredient calculated to produce a desired therapeutic effect (e.g., bronchodilation or relaxation of the airways). These terms are synonymous with the therapeutically effective amounts and amounts sufficient to achieve the stated goals of the methods disclosed herein.

As used herein, the terms “treat”, “treating”, and “treatment” have their ordinary and customary meanings, and include one or more of, ameliorating a symptom of an obstructive lung disease or condition in a subject, blocking or ameliorating a recurrence of a symptom of an obstructive lung disease or condition in a subject, decreasing in severity and/or frequency a symptom of an obstructive lung disease or condition in a subject. As used herein, “treatment” includes at least partially, and at least temporarily, relieving bronchoconstriction (or bronchospasm) or increasing bronchodilation, so that the patient or subject can breathe more easily. Treatment means ameliorating, blocking, reducing, decreasing or inhibiting by about 1% to about 100% versus a subject to which a pharmaceutical composition has not been administered. Preferably, the ameliorating, blocking, reducing, decreasing or inhibiting is about 100%, 99%, 98%, 97%, 96%, 95%, 90%, 80%, 70%, 60%, 50%, 40%, 30%, 20%, 10%, 5% or 1% versus a subject to which a pharmaceutical composition has not been administered.

As used herein, the terms “prevent”, “preventing”, and “prevention” have their ordinary and customary meanings, and include one or more of preventing a symptom of an obstructive lung disease or condition in a subject, blocking a recurrence of a symptom of an obstructive lung disease or condition in a subject, and decreasing in frequency a symptom of an obstructive lung disease or condition in a subject. As used herein, “prevention” includes at least partially, and at least temporarily, blocking bronchoconstriction (or bronchospasm) so that breathing is not inhibited in the patient or subject. The prevention may be protection of about 100%, 99%, 98%, 97%, 96%, 95%, 90%, 80%, 70%, 60%, 50%, 40%, 30%, 20%, 10%, 5% or 1% in the subject, versus a subject to which a pharmaceutical composition has not been administered. The prevention lasts at least about 1, 2, 3, 4, 5, 6, 7, 8, 9, 10, 11, 12, 13, 14, 15, 16, 17, 18, 19, 20, 21, 22, 23, or 24, or more, hours after administration of a pharmaceutical composition.

### EXAMPLES

The following examples are for the purpose of illustration of the invention only and are not intended to limit the scope of the present invention in any manner whatsoever.

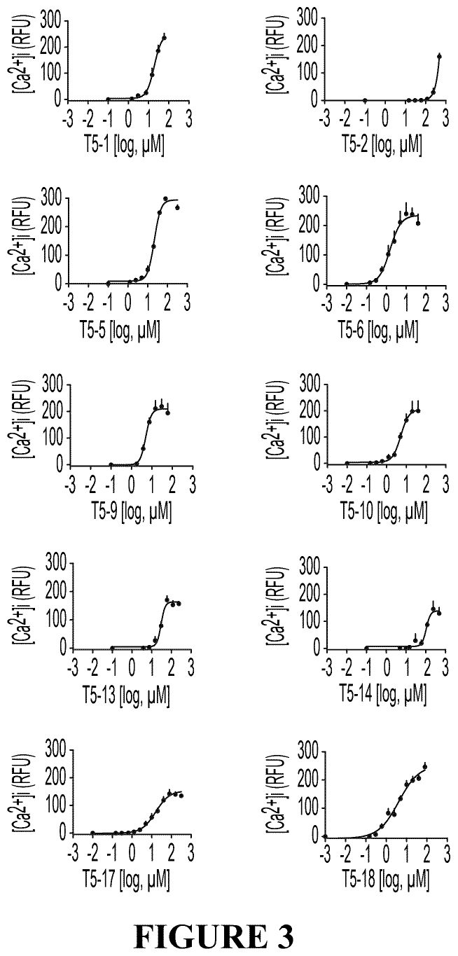

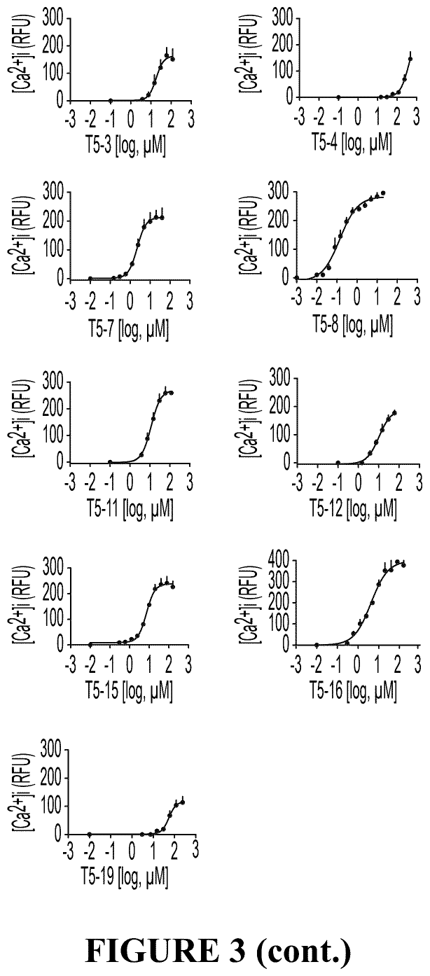

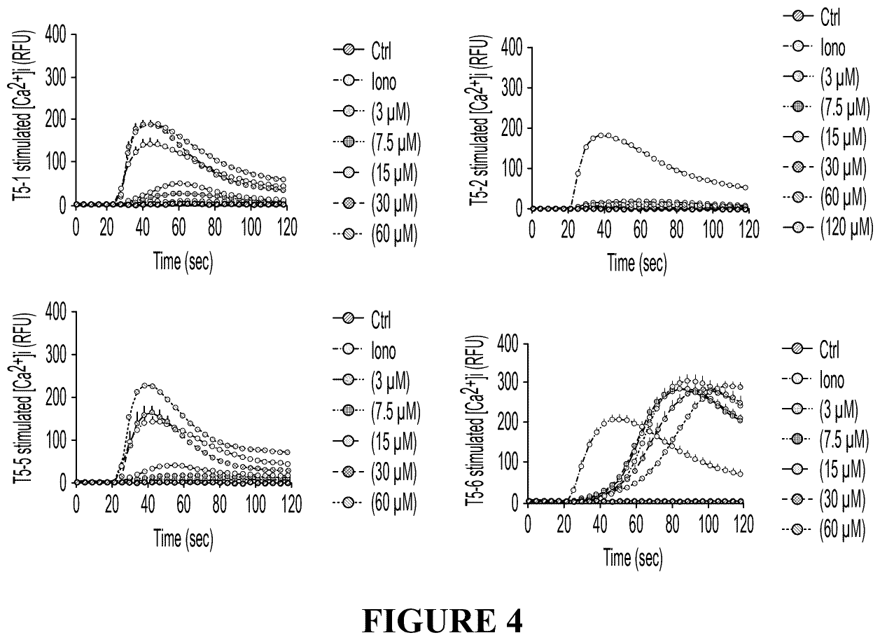

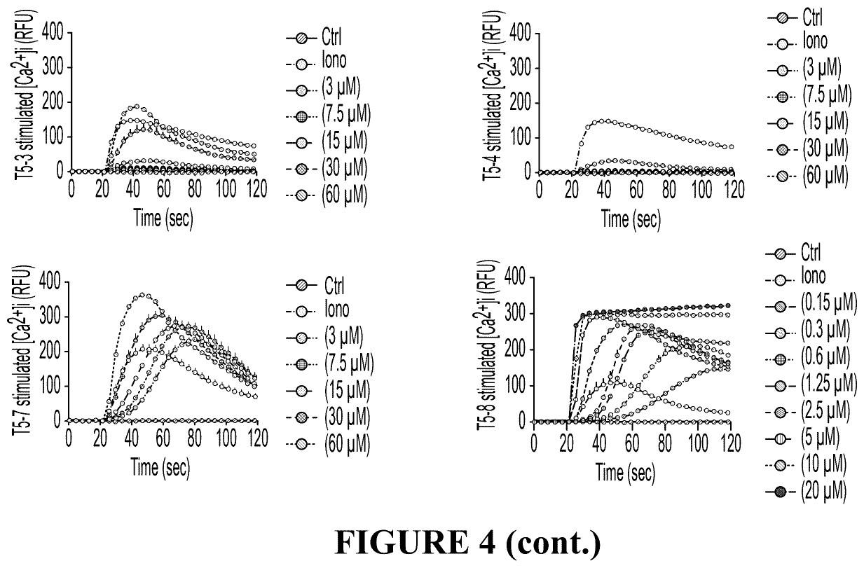

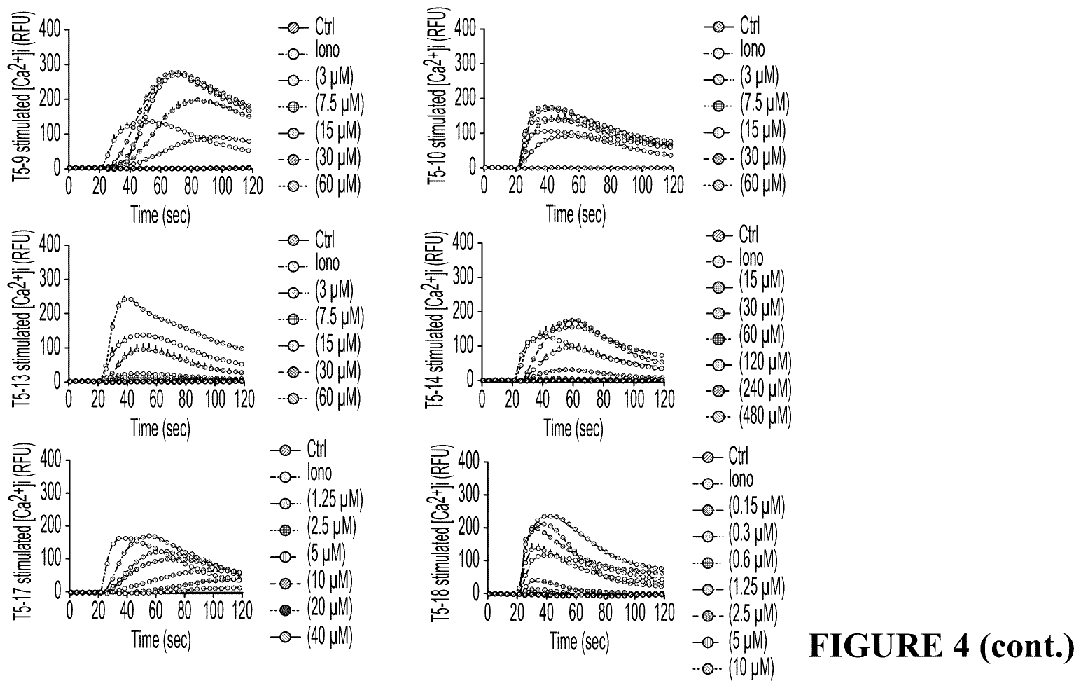

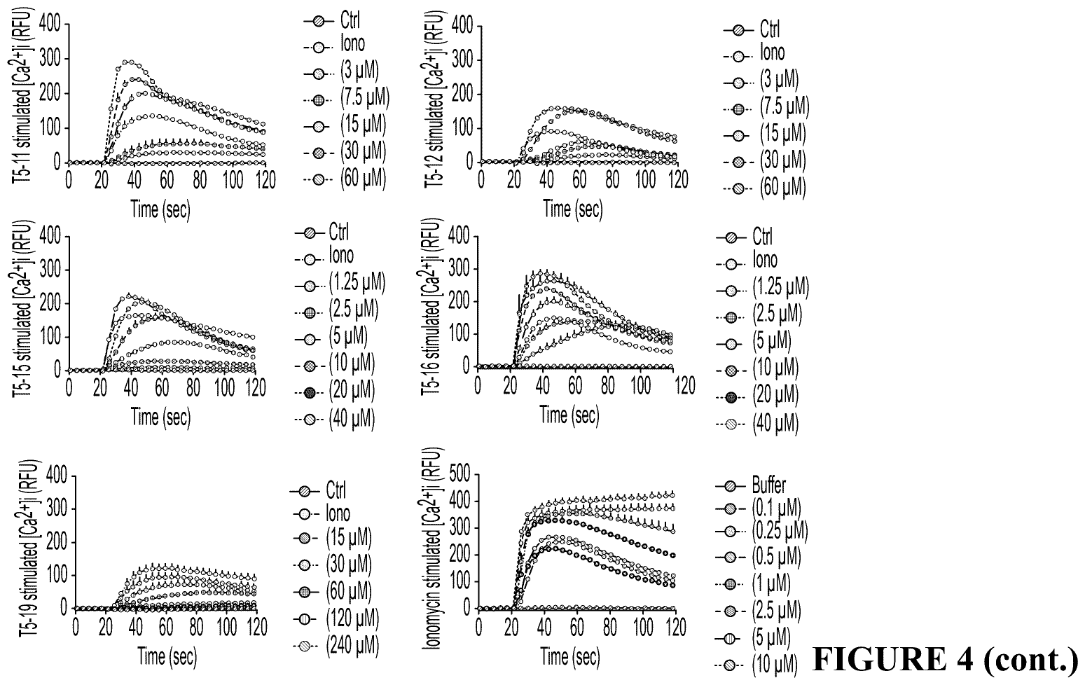

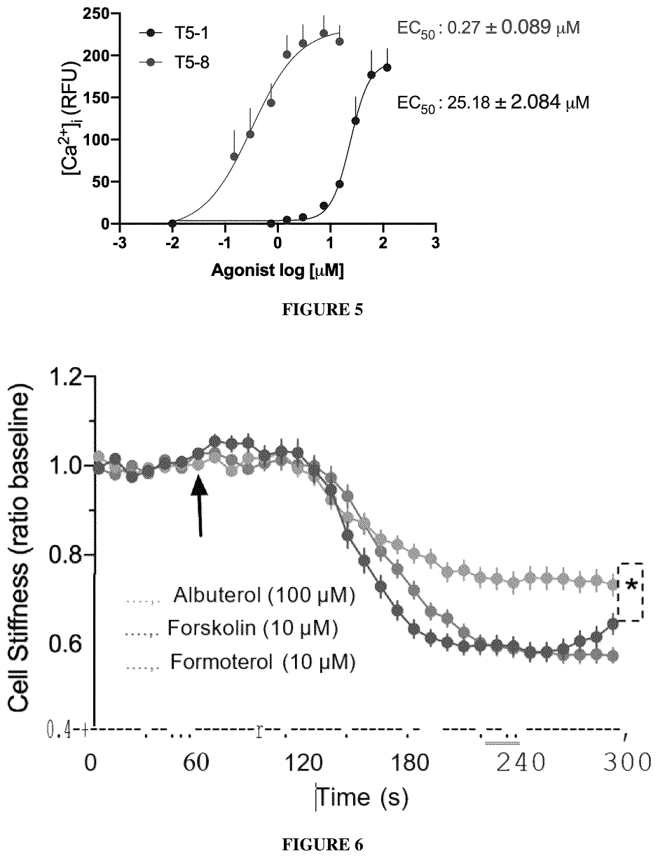

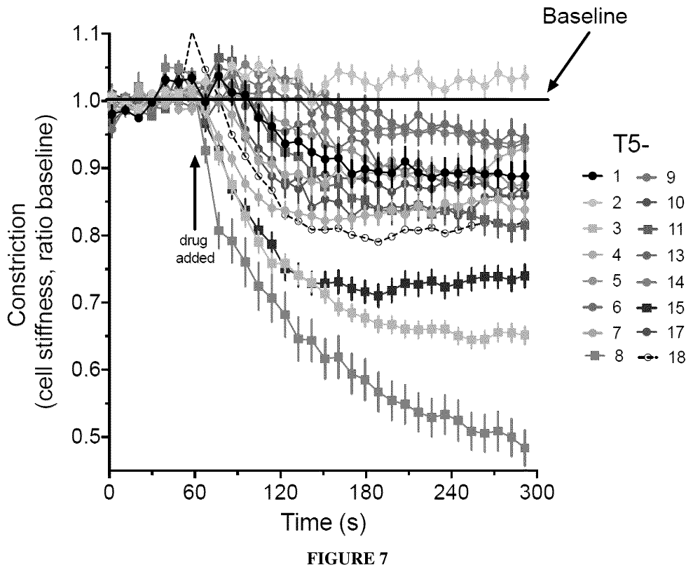

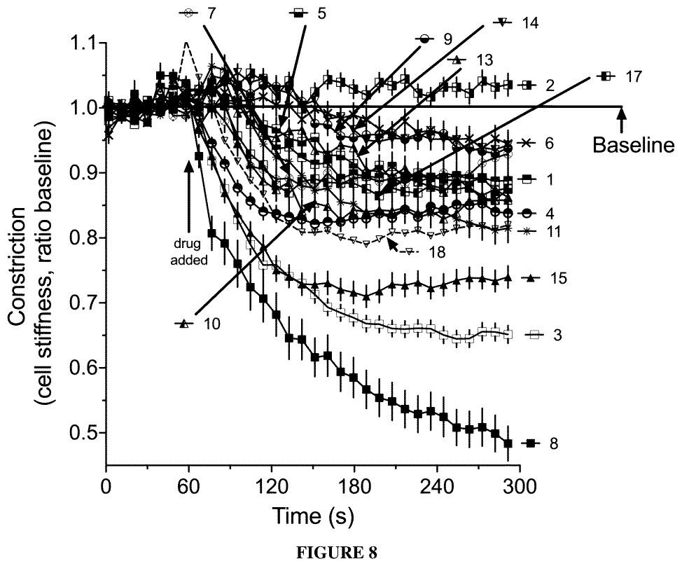

Cultured D₉ telomerase reverse transcriptase immortalized HASM (D9-HASM) cells were utilized for the [Ca²⁺]ᵢ screening assays. Cells were seeded onto 96 well plates and loaded with Fluo-4direct. The [Ca²⁺]ᵢ responses to multiple concentrations of potential agonists were determined as a measure of receptor activation. Control cells were exposed to the non-specific ionophore ionomycin at 2.0 μM (a maximal effective concentration, FIG. 4). Results from TAS2R agonist studies were normalized to the ionomycin response in order to compare the maximal responses (Rmax) between agonists. The concentration-response data were fit to a four component logistic regression model (sigmoid curve) using iterative non-linear regression methods to determine the EC₅₀ and Rmax. The structures of the previously identified agonist termed T5-1, and 18 derivatives (denoted as T5-2 through T5-19) are shown in FIG. 2. Representative [Ca²⁺]ᵢ responses to these compounds are shown in FIG. 4, and mean results from multiple concentration-response experiments are shown in FIG. 3. Table 1 summarizes the results in terms of potency (EC₅₀) and efficacy (Rmax), with statistical comparisons to T5-1.

To correlate the [Ca²⁺]ᵢ responses of the compounds to relevant physiologic function, we turned to measurements of single-cell mechanics in HASM cells using magnetic twisting cytometry (MTC) as previously described. Here, cultured primary HASM cells at passages 5-7 derived from a non-asthmatic donor lung were utilized. The decrease in cell stiffness evoked by a compound on the ferrimagnetically tagged HASM cells, twisted by a magnetic field, has been found to correlate with clinical airway relaxation. For these studies a single concentration of all agonists was utilized, recognizing that the 3 log differences in EC₅₀ values observed with the compounds would yield a range of relaxation responses consistent with the structure-activity relationships established in the [Ca²⁺]ᵢ assays, with some caveats (see below). Positive controls (FIG. 6) included the β-agonists albuterol (partial agonist), formoterol (full agonist) and forskolin, which increases cAMP by direct activation of adenylyl cyclase. The results from these studies are shown in FIG. 4 and Table 1.

TABLE 1
[Ca²⁺]ᵢ stimulation and smooth muscle relaxation responses
of HASM cells to the indicated compounds

| | | | Rmax | Relaxation |
|---|---|---|---|---|
| Agonist | Abbreviation | EC₅₀ (μM) | (% ionomycin) | (% basal) |
| 1,10-Phenanthroline | T5-1 | 29.7 ± 5.57 | 149 ± 10.23 | 10.8 ± 2.17^{#} |
| 2,2′-Bipyridine | T5-2 | N/A | 16.4 ± 4.48 | −3.2 ± 1.67 |
| 1,7-Phenanthroline | T5-3 | 24.9 ± 4.58 | 121 ± 16.7 | 34.8 ± 1.36^{#} |
| 4,7-Phenanthroline | T5-4 | N/A | 15.4 ± 3.37* | 15.3 ± 1.48^{#} |
| 2,9-Dimethyl-1,10- | T5-5 | 33.3 ± 6.40 | 143 ± 13.1 | 12.0 ± 1.47^{#} |
| phenanthroline | | | | |
| 4,7-Dimethyl-1,10- | T5-6 | 1.39 ± 0.19* | 147 ± 16.7 | 5.3 ± 1.95^{#} |
| phenanthroline | | | | |
| 5,6-Dimethyl-1,10- | T5-7 | 2.05 ± 0.20* | 163 ± 36.4 | 9.0 ± 2.19^{#} |
| phenanthroline | | | | |
| 1,10-Phenanthroline- | T5-8 | 0.12 ± 0.05* | 199 ± 20.2* | 50.2 ± 2.79^{#} |
| 5,6-dione | | | | |
| 1,10-Phenanthrolin-5- | T5-9 | 4.60 ± 0.42* | 176 ± 14.2 | 7.2 ± 1.95^{#} |
| amine | | | | |
| 5-Bromo-1,10- | T5-10 | 4.35 ± 0.71* | 182 ± 25.7 | 14.4 ± 1.93^{#} |
| phenanthroline | | | | |
| 5-Chrolo-1,10- | T5-11 | 11.1 ± 2.00 | 183 ± 7.3 | 18.0 ± 2.13^{#} |
| phenanthroline | | | | |
| 5-Methyl-1,10- | T5-12 | 12.1 ± 1.99 | 181 ± 24.6 | ND |
| phenanthroline | | | | |
| 5-Nitro-1,10- | T5-13 | 36.0 ± 3.85 | 134 ± 11.9 | 12.8 ± 2.00^{#} |
| phenanthroline | | | | |
| Pyrazino[2,3-f] | T5-14 | 146.6 ± 2.26* | 34.5 ± 15.1* | 6.1 ± 1.67^{#} |
| [1,10]phenanthroline | | | | |
| 5,6-Dimethoxy-1,10- | T5-15 | 7.48 ± 0.78* | 127 ± 16.1 | 26.7 ± 1.65^{#} |
| phenanthroline | | | | |
| 5,6-Diethyl-1,10- | T5-16 | 4.05 ± 0.80* | 183 ± 14.7 | ND |
| phenanthroline | | | | |
| 5,6-di(prop-1-en-2yl)- | T5-17 | 25.5 ± 7.06 | 113 ± 19.0 | 13.5 ± 2.59^{#} |
| 1,10-phenanthroline | | | | |
| 9,10- | T5-18 | 5.08 ± 1.37* | 153 ± 5.2 | 20.0 ± 2.00^{#} |
| Phenanthrenequinone | | | | |
| 4,4′-Dimethyl-2,2′- | T5-19 | 60.6 ± 9.13* | 58.9 ± 2.95* | ND |
| dipyridyl | | | | |

*EC₅₀ or Rmax different than T5-1, P < 0.05([Ca²⁺]ᵢ assays).
^{#}Relaxation greater than baseline, P < 0.05(MTC assays).
N/A, not applicable because of lack of curve fit
ND, The experiment was not done

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method for treating an obstructive lung disease in a patient in need thereof, comprising administering to the patient a compound having the formula: 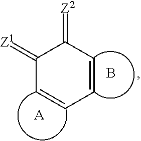
or a pharmaceutically acceptable salt thereof, wherein
ring A represents a carbocyclic or heterocyclic ring system;
ring b represents a carbocyclic or heterocyclic ring system;
Z¹ is O, S, or NR^{a}; wherein R^{a} is H, C₁₋₈alkyl, or C₁₋₈alkoxy; and
Z² is O, S, or NR^{b}; wherein R^{b} is H, C₁₋₈alkyl, or C₁₋₈alkoxy.

2. The method according to claim 1, wherein ring A is a heterocyclic ring system having the formula: 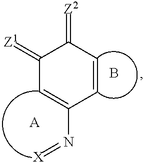
wherein
X is N or CR¹, wherein R¹ is selected from R^{a}, OR^{a}, N(R^{a})₂, F, Cl, Br, I, NO₂, CN, wherein R^{a} is in each case selected from H, C₁₋₈alkyl, C₂₋₈alkenyl; C₂₋₈alkynyl;

3. The method according to claim 2, wherein ring A defines the group: 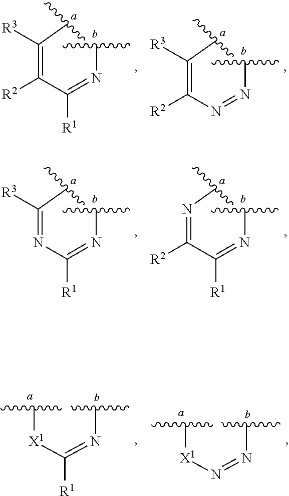
wherein wavy line “a” represent the point of attachment to the carbon ortho to the Z¹ bearing carbon, and wavy line “b” represents the point of attachment to the carbon para to the Z² bearing carbon;
X¹ is O, S, or NR⁴
R² is selected from R^{b}, OR^{b}, N(R^{b})₂, F, Cl, Br, I, NO₂, CN, wherein R^{b} is in each case selected from H, C₁₋₈alkyl, C₂₋₈alkenyl; C₂₋₈alkynyl;
R³ is selected from R^{c}, OR^{c}, N(R^{c})₂, F, Cl, Br, I, NO₂, CN, wherein R^{c} is in each case selected from H, C₁₋₈alkyl, C₂₋₈alkenyl; C₂₋₈alkynyl;
R⁴ is H or C₁₋₈alkyl, C₂₋₈alkenyl; C₂₋₈alkynyl;
wherein any two or more of R¹, R², R³, and R⁴ may together form a ring; and
wherein either R³ or R⁴ may form a ring with R^{a}.

4. The method according to claim 2, wherein the compound has the formula: 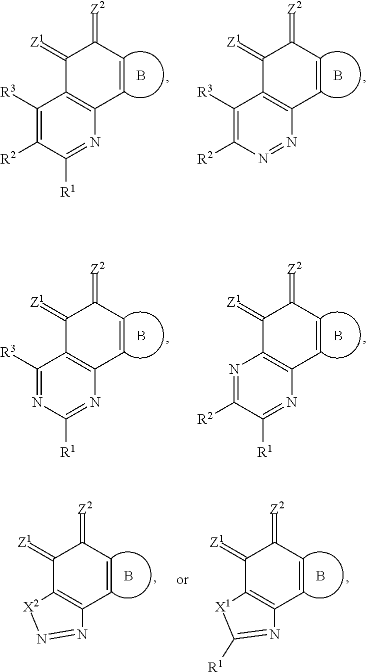
wherein X¹ is O, S, or NR⁴
R² is selected from R^{b}, OR^{b}, N(R^{b})₂, F, Cl, Br, I, NO₂, CN, wherein R^{b} is in each case selected from H, C₁₋₈alkyl, C₂₋₈alkenyl; C₂₋₈alkynyl;
R³ is selected from R^{c}, OR^{c}, N(R^{c})₂, F, Cl, Br, I, NO₂, CN, wherein R^{c} is in each case selected from H, C₁₋₈alkyl, C₂₋₈alkenyl; C₂₋₈alkynyl;
R⁴ is H or C₁₋₈alkyl, C₂₋₈alkenyl; C₂₋₈alkynyl;
wherein any two or more of R¹, R², R³, and R⁴ may together form a ring; and
wherein either R³ or R⁴ may form a ring with R^{a}.

5. The method according to claim 1, wherein ring B defines the group: 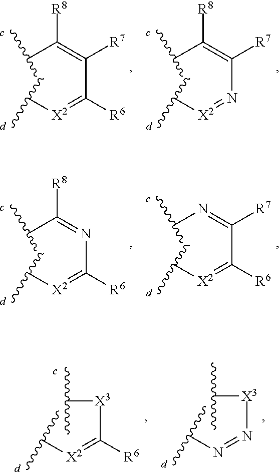
wherein wavy line “c” represent the point of attachment to the carbon ortho to the Z² bearing carbon, and wavy line “d” represents the point of attachment to the carbon para to the Z¹ bearing carbon;
X² is N or CR⁵;
X³ is N or CR⁹;
R⁵ is H or C₁₋₈alkyl, C₂₋₈alkenyl; C₂₋₈alkynyl;
R⁹ is H or C₁₋₈alkyl, C₂₋₈alkenyl; C₂₋₈alkynyl;
R⁶ is selected from R^{d}, OR^{d}, N(R^{d})₂, F, Cl, Br, I, NO₂, CN, wherein R^{d} is in each case selected from H, C₁₋₈alkyl, C₂₋₈alkenyl; C₂₋₈alkynyl;
R⁷ is selected from R^{e}, OR^{e}, N(R^{e})₂, F, Cl, Br, I, NO₂, CN, wherein R^{e} is in each case selected from H, C₁₋₈alkyl, C₂₋₈alkenyl; C₂₋₈alkynyl;
R⁸ is selected from R^{f}, OR^{f}, N(R^{f})₂, F, Cl, Br, I, NO₂, CN, wherein R^{f} is in each case selected from H, C₁₋₈alkyl, C₂₋₈alkenyl; C₂₋₈alkynyl;
wherein any two or more of R⁵, R⁶, R⁷, and R⁸ may together form a ring; and
wherein either R⁸ or R⁹ may form a ring with R^{b}.

6. The method according to claim 1, wherein the compound has the formula: 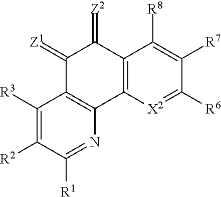
Wherein R¹ is selected from R^{a}, OR^{a}, N(R^{a})₂, F, Cl, Br, I, NO₂, CN, wherein R^{a} is in each case selected from H, C₁₋₈alkyl, C₂₋₈alkenyl; C₂₋₈alkynyl;
R² is selected from R^{b}, OR^{b}, N(R^{b})₂, F, Cl, Br, I, NO₂, CN, wherein R^{b} is in each case selected from H, C₁₋₈alkyl, C₂₋₈alkenyl; C₂₋₈alkynyl;
R³ is selected from R^{c}, OR^{c}, N(R^{c})₂, F, Cl, Br, I, NO₂, CN, wherein R^{c} is in each case selected from H, C₁₋₈alkyl, C₂₋₈alkenyl; C₂₋₈alkynyl;
R⁴ is H or C₁₋₈alkyl, C₂₋₈alkenyl; C₂₋₈alkynyl;
wherein any two or more of R¹, R², R³, and R⁴ may together form a ring; and
wherein R³ may form a ring with R^{a};
X² is N or CR⁵;
R⁵ is H or C₁₋₈alkyl, C₂₋₈alkenyl; C₂₋₈alkynyl;
R⁶ is selected from R^{d}, OR^{d}, N(R^{d})₂, F, Cl, Br, I, NO₂, CN, wherein R^{d} is in each case selected from H, C₁₋₈alkyl, C₂₋₈alkenyl; C₂₋₈alkynyl;
R⁷ is selected from R^{e}, OR^{e}, N(R^{e})₂, F, Cl, Br, I, NO₂, CN, wherein R^{e} is in each case selected from H, C₁₋₈alkyl, C₂₋₈alkenyl; C₂₋₈alkynyl;
R⁸ is selected from R^{f}, OR^{f}, N(R_{f})₂, F, Cl, Br, I, NO₂, CN, wherein R^{f} is in each case selected from H, C₁₋₈alkyl, C₂₋₈alkenyl; C₂₋₈alkynyl;
wherein any two or more of R⁵, R⁶, R⁷, and R⁸ may together form a ring; and
wherein R⁸ may form a ring with R^{b}.

7. The method according to claim 1, wherein Z¹ and Z² are both O.

8. The method according to claim 1, wherein the obstructive lung disease is asthma, chronic obstructive pulmonary disease, emphysema, bronchitis, cystic fibrosis, bronchiectasis, bronchiolitis, or allergic bronchopulmonary aspergillosis.

9. The method according to claim 1, wherein the compound is administered by nasal or buccal inhalation.

10. A pharmaceutical composition, comprising at least one compound according to claim 1, and a pharmaceutically acceptable excipient.

11. The pharmaceutical composition according to claim 10, comprising a fluorocarbon solvent.

12. The pharmaceutical composition according to claim 10, comprising a powdered matrix material.
